SEQUENCE LISTING (45 sequences)

SEQ ID NO: 1 - DNA, 93 bp, Homo sapiens
  misc_feature (1)..(93): Human proinsulin C-peptide CDS (Genbank NM_000207.2..gi109148525)
gaggcagaggacctgcaggtggggcaggtggagctgggcgggggccctggtgcaggcagcctgcagcccttggccctggaggggtccctgcag

SEQ ID NO: 2 - protein, 31 aa, Hominidae sp.
  MISC_FEATURE (1)..(31): Hominidae sp. proinsulin C-peptide  Human (Genbank P01308..gi124617), Chimpanzee (Genbank NP_001008996.1.. gi57113877), Orangutan (Genbank Q8HXV2..gi48428254), and Gorilla (Genbank AAM76640.1..gi23379796)
EAEDLQVGQVELGGGPGAGSLQPLALEGSLQ

SEQ ID NO: 3 - protein, 31 aa, Catarrhini sp.
  MISC_FEATURE (1)..(31): Catarrhini sp. proinsulin C-peptide  Rhesus macaque (Genbank 1006230A..gi223965), Crab-eating macaque (Genbank P30406..gi266373), and Green monkey (Genbank P30407..gi266362)
EAEDPQVGQVELGGGPGAGSLQPLALEGSLQ

SEQ ID NO: 4 - protein, 29 aa, Aotus trivirgatus
  MISC_FEATURE (1)..(29): Platyrrhini sp. proinsulin C-peptide  Northern night monkey (Genbank P67972..gi54037402)
EAEDLQVGQVELGGGSITGSLPPLEGPMQ

SEQ ID NO: 5 - protein, 26 aa, Bovidae-Caprinae sp.
  MISC_FEATURE (1)..(26): Bovidae-Caprinae sp. proinsulin C-peptide Cattle (Genbank P01317..gi124564), Water buffalo (Genbank BAE80772.1..gi89331178), European bison (Genbank 1312309A..gi225748), and Sheep (Genbank P01318..gi1708500)
EVEGPQVGALELAGGPGAGGLEGPPQ

SEQ ID NO: 6 - protein, 31 aa, Equidae sp.
  MISC_FEATURE (1)..(31): Equidae sp. proinsulin C-peptide  Horse (Genbank P01310..gi124616) and Przewalskii horse (Genbank AAB25818.1..gi299301)
EAEDPQVGEVELGGGPGLGGLQPLALAGPQQ

SEQ ID NO: 7 - protein, 29 aa, Sus scrofa
  MISC_FEATURE (1)..(29): Suidae sp. proinsulin C-peptide Sus scrofa (Genbank P01315..gi12643972)
EAENPQAGAVELGGGLGGLQALALEGPPQ

SEQ ID NO: 8 - DNA, 93 bp, Felis catus
  misc_feature (1)..(93): Felis catus proinsulin C-peptide CDS (Genbank AB043535.1..gi18376636)
gaggcggaggacctccaggggaaggacgcggagctgggggaggcgcctggcgccggcggcctgcagccctcggccctggaggcgcccctgcag

SEQ ID NO: 9 - protein, 31 aa, Felis catus
  MISC_FEATURE (1)..(31): Felidae sp. proinsulin C-peptide  Felis catus (Genbank P06306..gi90110828)
EAEDLQGKDAELGEAPGAGGLQPSALEAPLQ

SEQ ID NO: 10 - DNA, 93 bp, Canis familiaris
  misc_feature (1)..(93): Canis familiaris proinsulin C-peptide CDS (Genbank V00179.1..gi994)
gaggtggaggacctgcaggtgagggacgtggagctggccggggcgcctggcgagggcggcctgcagcccctggccctggagggggccctgcag

SEQ ID NO: 11 - protein, 31 aa, Canis familiaris
  MISC_FEATURE (1)..(31): Canidae sp. proinsulin C-peptide  Canis familiaris (Genbank P01321..gi124575)
EVEDLQVRDVELAGAPGEGGLQPLALEGALQ

SEQ ID NO: 12 - protein, 31 aa, Oryctolagus cuniculus
  MISC_FEATURE (1)..(31): Lagomorpha sp. proinsulin C-peptide Oryctolagus cuniculus (Genbank P01311..gi1352467)
EVEELQVGQAELGGGPGAGGLQPSALELALQ

SEQ ID NO: 13 - protein, 29 aa, Mus sp.
  MISC_FEATURE (1)..(29): Mouse sp. proinsulin type 1 C-peptide  House mouse (Genbank P01325..gi124511) and Western wild mouse (Genbank AAB60473.1..gi497063)
EVEDPQVEQLELGGSPGDLQTLALEVARQ

SEQ ID NO: 14 - protein, 31 aa, Mus sp.
  MISC_FEATURE (1)..(31): Mouse sp. proinsulin type 2 C-peptide  House mouse (Genbank P01326..gi124519), Western wild mouse (Genbank CAJ76270.1..gi86989480, and Ryukyu mouse (Genbank ABB89749.1..gi82749730)
EVEDPQVAQLELGGGPGAGDLQTLALEVAQQ

SEQ ID NO: 15 - protein, 29 aa, Mus caroli
  MISC_FEATURE (1)..(29): Ryukyu mouse proinsulin type 1 C-peptide  Mus caroli (Genbank ABB89745.1..gi82749722)
EVEDPQVEQLELGGSPGDLQTLALEVARE

SEQ ID NO: 16 - protein, 31 aa, Apodemus semotus
  MISC_FEATURE (1)..(31): Taiwan field mouse proinsulin type 1 C-peptide Apodemus semotus (Genbank ABB89744.1..gi82749720)
EVEDPQVEQLELGGAPGTGDLETLALEVARQ

SEQ ID NO: 17 - protein, 31 aa, Rattus norvegicus
  MISC_FEATURE (1)..(31): Norway rat proinsulin type 1 C-peptide (Genbank AAA41442.1..gi204957)
EVEDPQVPQLELGGGPEAGDLQTLALEVARQ

SEQ ID NO: 18 - protein, 31 aa, Rattus norvegicus
  MISC_FEATURE (1)..(31): Rat sp. proinsulin type 2 C-peptiide  Rattus norvegicus (Genbank AAA41443.1..gi204959) and Apodemus semotus (Genbank ABB89748.1..gi82749728)
EVEDPQVAQLELGGGPGAGDLQTLALEVARQ

SEQ ID NO: 19 - protein, 31 aa, Rattus losea
  MISC_FEATURE (1)..(31): Lesser rice-field rat proinsulin type 1 C-peptide (Genbank ABB89743.1..gi82749718)
EVEDPQVPQLELGGSPEAGDLQTLALEVARQ

SEQ ID NO: 20 - protein, 31 aa, Rattus losea
  MISC_FEATURE (1)..(31): Lesser rice-field rat proinsulin type 2 C-peptide (Genbank ABB89747.1..gi82749726)
EVEDPQVAQQELGGGPGAGDLQTLALEVARQ

SEQ ID NO: 21 - protein, 31 aa, Niviventer coxingi
  MISC_FEATURE (1)..(31): Coxing′s white-bellied rat proinsulin type 1 C-peptide (Genbank ABB89746.1..gi82749724)
EVEDPQVAQLELGEGPEAGDLQTLALEVARQ

SEQ ID NO: 22 - protein, 31 aa, Niviventer coxingi
  MISC_FEATURE (1)..(31): Coxing′s white-bellied rat proinsulin type 2 C-peptide (Genbank ABB89750.1..gi82749732)
EVEDPQVPQLELGGGPGTGDLQTLALEVARQ

SEQ ID NO: 23 - protein, 31 aa, Rodentia sp.
  MISC_FEATURE (1)..(31): Rodentia sp. proinsulin C-peptide (Genbank P21563..gi124615)
EVEDPQVGQVELGAGPGAGSEQTLALEVARQ

SEQ ID NO: 24 - protein, 31 aa, Spermophilus tridecemlineatus
  MISC_FEATURE (1)..(13): Thirteen-lined ground squirrel proinsulin C-peptide (Genbank Q91XI3..gi29427536)
EVEEQQGGQVELGGGPGAGLPQPLALEMALQ

SEQ ID NO: 25 - protein, 31 aa, Meriones unguiculatus
  MISC_FEATURE (1)..(31): Mongolian gerbil proinsulin C-peptide (Genbank ABB89751.1..gi82749734)
GVEDPQMPQLELGGSPGAGDLQALALEVARQ

SEQ ID NO: 26 - protein, 31 aa, Psammomys obesus
  MISC_FEATURE (1)..(31): Fat sand rat proinsulin C-peptide (Genbank Q62587..gi2497407)
GVDDPQMPQLELGGSPGAGDLRALALEVARQ

SEQ ID NO: 27 - protein, 31 aa, Microtus kikuchii
  MISC_FEATURE (1)..(31): Taiwan vole proinsulin C-peptide (Genbank ABB89752.1..gi82749736)
GVEDPQVAQLELGGGPGAGDLQTLALEVAQQ

SEQ ID NO: 28 - protein, 31 aa, Cricetulus longicaudatus
  MISC_FEATURE (1)..(31): Long-tailed hamster proinsulin C-peptide (Genbank P01313..gi124588)
GVEDPQVAQLELGGGPGADDLQTLALEVAQQ

SEQ ID NO: 29 - protein, 31 aa, Cavia porcellus
  MISC_FEATURE (1)..(31): Cavy proinsulin type 1 C-peptide (Genbank P01329..gi124581)
ELEDPQVEQTELGMGLGAGGLQPLALEMALQ

SEQ ID NO: 30 - protein, 29 aa, Cavia porcellus
  MISC_FEATURE (1)..(29): Cavy proinsulin type 2 C-peptide (Genbank)740063A..gi229473
ELEDPQVEQTELGMGLGAGGLQPLQGALQ

SEQ ID NO: 31 - protein, 31 aa, Chinchilla brevicaudata
  MISC_FEATURE (1)..(31): Chinchilla proinsulin C-peptide (Genbank P01327..gi83303940)
ELEDPQVGQADPGVVPEAGRLQPLALEMTLQ

SEQ ID NO: 32 - protein, 29 aa, Octodon degus
  MISC_FEATURE (1)..(29): Chilean brush tail rat proinsulin C-peptide (Genbank P17715..gi124651)
ELEDLQVEQAELGLEAGGLQPSALEMILQ

SEQ ID NO: 33 - protein, 28 aa, Gallus gallus
  MISC_FEATURE (1)..(29): Chicken and Pigeon proinsulin C-peptide Gallus gallus (Genbank P67970..gi54037398) and Columba livia (Genbank AAP45955.1..gi32891835 and AAP45983.1..gi32891891)
DVEQPLVSSPLRGEAGVLPFQQEEYEKV

SEQ ID NO: 34 - protein, 28 aa, Meleagris gallopavo
  MISC_FEATURE (1)..(28): Turkey proinsulin C-peptide (Genbank AAP45962.1..gi32891849 and AAP45990.1..gi32891905)
DVEQPLVGSPLRGEAGVLPFQQEEYEKV

SEQ ID NO: 35 - protein, 28 aa, Anas sp.
  MISC_FEATURE (1)..(28): Duck proinsulin C-peptide A. platyrhynchos var. Mallard (Genbank P01333..gi124547); A. platyrhynchos var. Peking (Genbank AAP45950.1..gi32891825 & AAP45978.1..gi32891881); A. crecca (Genbank AAP45949.1..gi32891823 & AAP45977.1..gi32891879)
DVEQPLVNGPLHGEVGELPFQHEEYQKV

SEQ ID NO: 36 - protein, 28 aa, Xenopus laevis
  MISC_FEATURE (1)..(28): African clawed frog proinsulin type 1 C-peptide (Genbank P12706..gi124513)
DMEQALVSGPQDNELDGMQLQPQEYQKM

SEQ ID NO: 37 - protein, 28 aa, Xenopus laevis
  MISC_FEATURE (1)..(28): African clawed frog proinsulin type 2 C-peptide (Genbank P12707..gi124526)
DIEQAQVNGPQDNELDGMQFQPQEYQKM

SEQ ID NO: 38 - protein, 5 aa, Artificial
  C-peptide residues 27-31
EGSLQ

SEQ ID NO: 39 - protein, 12 aa, Artificial
  C-peptide residues 20-31 from human sequence
SLQPLALEGSLQ

SEQ ID NO: 40 - protein, 22 aa, Artificial
  C22 C-peptide residues 10-31 from human sequence
VELGGGPGAGSLQPLALEGSLQ

SEQ ID NO: 41 - protein, 9 aa, Artificial
  Cmid C-peptide residues 11-19
ELGGGPGAG

SEQ ID NO: 42 - protein, 13 aa, Artificial
  N13 C-peptide residues 1-13
EAEDLQVGQVELG

SEQ ID NO: 43 - protein, 5 aa, Artificial
  AGSLQ C-peptide residues 27-31 (E27A)
AGSLQ

SEQ ID NO: 44 - protein, 5 aa, Artificial
  DGSLQ C-peptide residues 27-31 (E27D)
DGSLQ

SEQ ID NO: 45 - protein, 5 aa, Artificial
  SQLEG C-peptide residues 27-31 (scrambled)
SQLEG

(19) United States
(12) Patent Application Publication
(10) Pub. No.: US 2011/0020471 A1
(43) Pub. Date: Jan. 27, 2011

(54) ERYTHROCYTE ATP-RELEASE MODULATORS

(75) Inventors: Dana Spence, Howell, MI (US); Gavin Edmund Reid, Mason, MI (US)

Correspondence Address: HARNESS, DICKEY & PIERCE, P.L.C., P.O. BOX 828, BLOOMFIELD HILLS, MI, 48303, US

(73) Assignees: Wayne State University, Detroit, MI (US); Board of Directors of Michigan State University, East Lansing, MI (US)

(21) Appl. No.: 12/565,536

(22) Filed: Sep. 23, 2009

Related U.S. Application Data
(63) Continuation of application No. PCT/US2008/003801, filed on Mar. 22, 2008.
(60) Provisional application No. 60/919,952, filed on Mar. 23, 2007.

(51) Int. Cl. A61K 38/28; A61K 31/522; A61K 31/55; A61K 33/24; C07K 14/00; A61P 9/08; A61P 3/10; A61P 3/00; A61P 7/06; A61P 15/00

(52) U.S. Cl. 424/655; 514/5.9; 514/6.4; 514/6.5; 514/263.36; 514/263.31; 514/217.11; 530/324

(57) ABSTRACT

Erythrocyte ATP-release modulators and composition and methods for their use to potentiate serum glucose clearance and vasodilation; methods for prophylactic or palliative therapy of glucose processing or vascular disorders; processes for preparing the modulators and compositions comprising them; and kits therefor.

### CROSS-REFERENCE TO RELATED APPLICATIONS

This application is a continuation of International Application No. PCT/US2008/003801, filed Mar. 22, 2008, which claims the benefit of U.S. Provisional Application No. 60/919,952, filed Mar. 23, 2007. The entire disclosures of the above applications are incorporated herein by reference.

### GOVERNMENT RIGHTS

This invention was made with Governmental Support by NIH Grant No. HL 073942 awarded by the National Institutes of Health. The Government has certain rights in this invention.

### BACKGROUND

The present disclosure relates to compounds, compositions and methods useful to treat vascular conditions or glucose processing disorders.

A growing prevalence of vascular conditions and glucose processing disorders has resulted in a drive to develop new or improved treatments. As a result the pharmaceutical industry and academic research community are investing millions of dollars to provide advanced therapies for such conditions. Examples of these conditions include diabetes mellitus types 1 and 2, gestational diabetes, metabolic syndrome, hypertension, gestational hypertension, peripheral vascular diseases, chronic venous insufficiency, Raynaud's disease, and such conditions in other disorders, e.g., Raynaud's involvement in scleroderma, lupus, Sjögren's syndrome, or rheumatoid arthritis.

While small molecule pharmaceuticals are available, and more are being tested for such conditions, biomolecular pharmaceuticals are also useful; in the case of diabetes, the classic example is insulin. Insulin acts on glucose transporter type 4, a cell surface protein expressed on adipose and striated muscle cells. However, cellular resistance to insulin is increasingly encountered, e.g., in metabolic syndrome and type 2 diabetes. As a result, it would be beneficial to provide pharmaceuticals or biopharmaceuticals that can act as alternatives to or supplements for insulin administration in glucose processing disorders.

In regard to vascular conditions, synthetic agents that promote vasodilation are widely used, e.g.: hydralazine, isosorbide, minoxidil, nitroglycerin, nitroprusside, alpha-adrenergic blockers such as phentolamine, and cGMP-specific phosphodiesterase type 5 (PDE5) inhibitors such as sildenafil. Many such drugs are metabolized by the liver and can present significant side effects. As a result, it would be beneficial to provide a less toxic agent that could act as an alternative or supplements to such drugs.

In regard to vasodilation, recent research has identified some of the putative mechanisms that can cause this phenomenon in healthy states. For example, RBCs have been found to release ATP upon distortion of their cell membranes, and it has been reported that ATP release by RBCs is capable of stimulating nitric oxide production by vascular endothelial cells, thereby causing release of this vasodilation signal to vascular smooth muscle. See, R. S. Sprague et al., in Am. J. Physiol. Heart Circ. Physiol. 271(6):H2717-H2722 (December 1996); R. S. Sprague et al., in Exp. Biol. & Med. 226(5):434-439 (May 2001); and R. S. Sprague et al., in Am. J. Physiol. Heart Circ. Physiol. 285(2): H693-H700 (August 2003) (e-Publ. Apr. 10, 2003; doi:10.1152/ajpheart.01026.2002).

Yet, in diabetes and some other conditions, RBC plasma membranes are altered to have a more rigid, less flexible rheology. See, M. Garnier et al., in Metabolism 30(8):794-98 (1990). For example, the distorted morphology of RBCs in sickle cell anemia also results in membrane rigidity. See, e.g., R. Messmann et al., in Blood 75(8):1711-17 (Apr. 15, 1990). Similar RBC membrane rigidity is found in malaria, as well. See, J. P. Shelby et al., in PNAS USA 100(25):14618-14622 (Dec. 9, 2003) (e-Publ. Nov. 24, 2003; doi: 10.1073/pnas.2433968100). RBC membrane rigidity is likewise found in chronic fatigue syndrome, and in obesity. See, R. S. Richards et al., in Arch. Med. Res. 38(1):94-98 (January 2007); and R. Cazzola et al., in J. Lipid Res. 45:1846-1851 (October 2004) (e-Publ. Jul. 1, 2004; doi:10.1194/jlr.M300509-JLR200). Such conditions can thereby also result in impaired systemic vasodilation.

One biomolecular agent that has been described as potentially useful for treatment of diabetes pathology is the proinsulin C-peptide. See, PCT Pub. No. WO 2005/039627, Ekberg et al., published May 6, 2005; and A. A. F. Sima, in Rev. Diabet. Stud. 1(2):55-57 (Summer 2004) (e-Publ. Aug. 10, 2004; doi: 10.1900/RDS.2004.1.55). Yet, results of bioactivity tests of the proinsulin C-peptide have been inexplicably inconsistent. See, e.g., P. N. Shashkin et al., C-peptide does not alter carbohydrate metabolism in isolated mouse muscle, Am. J. Physiol. 272(2 Pt 1):E245-47 (February 1997); E. Polska et al., C-peptide does not affect ocular blood flow in patients with type 1 diabetes, Diabetes Care 29(9):2034-38 (September 2006); and B. J. Hoogwerf et al., Infusion of synthetic human C-peptide does not affect plasma glucose, serum insulin, or plasma glucagon in healthy subjects, Metabolism 35(2):122-25 (February 1986).

Therefore, it would be advantageous to provide compounds and compositions that can offer reduced toxicity while exhibiting effectiveness as alternatives to or supplements for small molecule vasodilators in vascular disorders and/or as alternatives to or supplements for insulin administration in glucose processing disorders.

### SUMMARY

The present technology provides compounds and compositions for the treatment or prevention of glucose metabolism disorders, vascular disorders, disorders associated with reduced erythrocyte ATP release, and associated diseases and disorders. Compositions of the present technology comprise erythrocyte ATP-release modulators, e.g., compounds that modulate the release of ATP by red blood cells (RBCs). Compounds useful herein include those selected from the group consisting of pentoxifylline, lisofylline, epoxidated arachidonic acids, and salts and esters thereof; mixtures of C-peptide, or a fragment thereof, and a source of a pharmaceutically acceptable polyvalent metal cation; complexes comprising a C-peptide, or a fragment thereof, and a polyvalent metal cation; and mixtures thereof.

In one aspect, the present technology provides compositions in unit dosage form comprising a pharmaceutically acceptable complex of a C-peptide or a fragment thereof with a polyvalent metal cation complex, and a pharmaceutically acceptable carrier. Preferably, the polyvalent metal cation is a pharmaceutically acceptable metal cation, such as divalent or trivalent metal cations or a combination thereof. Such cations include divalent Mg, Ca, Sr, Ba, Ge, or Sn cations; trivalent Al, Ga, In, or Bi cations; di- or trivalent transition metal cations; or di- or trivalent La, Ce, Pr, Nd, Pm, Sm, Eu, Gd, Tb, Dy, Ho, Er, Tm, Yb, or Lu cations; polyvalent V, Cr, Mn, Fe, Co, Ni, Cu, Zn, Mo, Ag, Pt, or Au cations; and combinations thereof. Methods and kits for forming such complexes are also provided.

Methods include those for modulating erythrocyte ATP-release in a human or other animal subject in need thereof, comprising administering the subject a composition comprising an erythrocyte ATP-release modulator, such as an active selected from the group consisting of pentoxifylline, lisofylline, epoxidated arachidonic acids, and salts and esters thereof; mixtures of C-peptide, or a fragment thereof, and a source of a pharmaceutically acceptable polyvalent metal cation; complexes comprising a C-peptide, or a fragment thereof, and a polyvalent metal cation; and mixtures thereof. Also provided are methods for promoting vasodilation in a human or other animal subject in need thereof, comprising administering to the subject a safe and effective amount of an erythrocyte ATP-release modulator. In various embodiments, methods include those for promoting glucose clearance or vasodilation in a human or animal subject, comprising administering to the subject a therapeutically effective amount of a pharmaceutically acceptable C-peptide (or fragment)/metal cation complex in which the metal cation comprises a pharmaceutically acceptable metal cations, preferably a M(II) or M(III) cation. In some embodiments, the erythrocyte ATP-release modulator compound or composition can be used to achieve both a vascular and a glucose processing benefit. Also provided are regimens for treating diabetes mellitus in a human or other animal subject comprising administering to the subject a glucose metabolism modulator and erythrocyte ATP-release modulator, wherein said erythrocyte ATP-release modulator is effective to reduce the level of the glucose metabolism modulator needed to effect glucose control in the subject, extend the duration of efficacy of the glucose metabolism modulator in the subject, or both.

It has been found that compounds, compositions and methods of the present technology afford advantages over treatment methods among those known in the art. Such advantages include one or more of enhanced efficacy in the treatment of glucose metabolism disorders, enhanced efficacy in the treatment of vascular conditions, potentiation of insulin and other glucose metabolism modulators, and reduced toxicity or other side effects. For example, compounds, compositions and methods of the present technology may offer reduced toxicity while exhibiting effectiveness as alternatives to, or supplements, for small molecule vasodilators in vascular disorders and as alternatives to, or supplements, for insulin or hypoglycemic agents in glucose processing disorders. Other benefits will be apparent to one of skill in the art.

### FIGURES

The drawings described herein are for illustration purposes only and are not intended to limit the scope of the present disclosure in any way.

FIG. 1 presents bar chart results of determination of ATP release from rabbit red blood cells (RBCs) subjected to flow in the presence and absence of a freshly prepared C-peptide preparation.

FIG. 2 presents bar chart results of determination of ATP release from diabetic human RBCs: 63.6±12.6 nM ATP release at 0 hours; and 256.1±38.7 nM ATP release after 6 hours of incubation with a freshly prepared C-peptide preparation. Error bars are ±SEM (n=7).

FIGS. 3A and 3B present electrospray ionization mass spectrograms (ESI-MS) of a freshly made C-peptide preparation (3A) and a C-peptide preparation after refrigeration for 24 hours (3B).

FIGS. 4A and 4B present an ESI-MS of C-peptide incubated with iron II (4A); and a chart (4B) of results of ATP release by rabbit RBCs incubated with iron II (390.6±6.3 nM) and with iron II/C-peptide (1000±23.0 nM). Error bars are ±SEM.

FIGS. 5A and 5B present an ESI-MS of C-peptide incubated with chromium III (5A); and a chart (5B) of result of ATP release by rabbit RBCs incubated with chromium III (303.6±13.0 nM) and with chromium III/C-peptide (743.7±54.1 nM).

FIG. 6 presents a chart of results of ATP release by human RBCs (537.3±7.2 nM) incubated with C-peptide and iron II (729.3±49.7 nM) or with C-peptide and chromium III (1292±61.4 nM) after 72 hours.

It should be noted that the figures set forth herein are intended to exemplify the general characteristics of the compounds, compositions, and methods among those of this technology, for the purpose of the description of such embodiments herein. These figures may not precisely reflect the characteristics of any given embodiment, and are not necessarily intended to define or limit specific embodiments within the scope of this technology.

### DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. The following definitions and non-limiting guidelines must be considered in reviewing the description of the technology set forth herein.

The headings (such as “Background” and “Summary,”) and sub-headings (such as “Compositions”) used herein are intended only for general organization of topics within the disclosure of the technology, and are not intended to limit the disclosure of the technology or any aspect thereof. In particular, subject matter disclosed in the “Field” and “Background” may include aspects of technology within the scope of one or more inventions, and may not constitute a recitation of prior art. Subject matter disclosed in the “Summary” is not an exhaustive or complete disclosure of the entire scope of the technology or any embodiments thereof.

The citation of references herein does not constitute an admission that those references are prior art or have any relevance to the patentability of the technology disclosed herein. Any discussion of the content of references cited in the Introduction is intended merely to provide a general summary of assertions made by the authors of the references, and does not constitute an admission as to the accuracy of the content of such references. All references cited in the Description section of this specification are hereby incorporated by reference in their entirety.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific Examples are provided for illustrative purposes of how to make, use and practice the compositions and methods of this technology and, unless explicitly stated otherwise, are not intended to be a representation that given embodiments of this technology have, or have not, been made or tested.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

The present technology provides compounds, compositions and methods for the treatment or prevention of various disorders in humans or other animals. Accordingly, specific compounds and compositions to be used in this technology must, accordingly, be pharmaceutically acceptable. As used herein, such a “pharmaceutically acceptable” component is one that is suitable for use with humans and/or animals without undue adverse side effects (such as toxicity, irritation, and allergic response) commensurate with a reasonable benefit/risk ratio.

The present technology provides erythrocyte ATP-release modulators. Erythrocyte ATP-release modulators among those useful herein are compounds or complexes that are operable to increase the ability of erythrocytes to release ATP. In various embodiments, such modulators effect an increase in ATP release by erythrocytes. Such modulators may effect an increase in ATP release when erythrocytes are deformed or otherwise subjected to physical force. Alternatively, such modulators may effect an increase in ATP release by erythrocytes that are not subjected to a deformation force.

Without limiting the mechanism, function or utility of the present technology, in various embodiments, contacting erythrocytes with an erythrocyte ATP-release modulator may increase glucose uptake by the erythrocyte, with a concomitant increase in glycolysis and adenocyclase activity, thereby generating ATP. Thus, for example, an erythrocyte ATP-release modulator can be employed to increase serum glucose clearance. The erythrocyte ATP-release modulator may also increase vasodilation or the vasodilation potential of RBCs.

Though not bound by theory, it is believed that the effect on glucose uptake may be mediated by an interaction between the C-peptide complex and the type 1 facilitative glucose transporter (GLUT1) expressed on the RBC plasma membrane. Thus, in some embodiments hereof, a C-peptide, C-peptide fragment, C-peptide/fragment complex or other erythrocyte ATP-release modulator may increase glucose uptake by cells expressing GLUT1.

GLUT 1 is expressed in many fetal and neonatal tissues, but in adults it is mainly found on erythrocytes and on endothelial and epithelial barrier tissues including, e.g., the blood-brain barrier, the blood-cerebrospinal fluid barrier, the retinal capillary endothelium, the retinal pigment epithelium, and others. Therefore, in some embodiments hereof, an erythrocyte ATP-release modulator can be used to increase glucose uptake in endothelial and epithelial barrier tissues. Such erythrocyte response modulators are particularly useful in some embodiments for enhancing RBC ATP release, or ATP release potential, in conditions that present increased RBC rigidity. Though not bound by theory, it is believed that these compounds may act in some embodiments by causing an increase in RBC plasma membrane flexibility.

Erythrocyte ATP-release modulators include substances that, following incubation with red blood cells, are found by assay to cause a significant increase in ATP-release-under-deformation therefrom as identified by an at least 2%, preferably at least about 5%, more preferably at least about 10%, increase in bioluminescence from treated RBCs compared to that from untreated RBCs under identical conditions. The assay involves measuring ATP-release from treated and untreated red blood cells that are separately tested in a cell-compatible fluid containing luciferin and luciferase, under luciferase-operative conditions, wherein the ATP-release level is measured when plasma membranes of the cells are physically deformed without lysis. A bioluminescence assay useful for this purpose is disclosed in PCT Pub. No WO 2008/118390, Spence, published Oct. 2, 2008, incorporated by reference herein. In such an assay, a stream of the RBC suspension can be passed through a flow channel that contains a deflection or a constriction whose internal dimension is about 2-20 μm, or it can be passed through a flow channel of about 100 μm or smaller internal dimension, e.g., 50-100 μm, through which the flow itself provides sufficient shear stress to allow ATP release without lysis. It should be noted, however, that while the release of ATP by erythrocytes under deformation may be used to identify erythrocyte ATP-release modulators, the mechanism, function or utility of such modulators is not limited or otherwise defined by the ability of the modulators to affect the release of ATP by erythrocytes in clinical applications.

In various embodiments, erythrocyte ATP-release modulators are selected from the group consisting of pentoxifylline (1-(5-oxohexyl)theobromine), lisofylline (1-(5-hydroxyhexyl)theobromine), epoxidated arachidonic acids (e.g., 5,6-epoxy-eicosatrienoic acid), and salts and esters thereof; C-peptides and fragments thereof; a physically separate or mixed combination of C-peptide or fragment thereof and a source of a pharmaceutically acceptable polyvalent metal cation; complexes comprising a C-peptide or fragment thereof and a polyvalent metal cation; and combinations, e.g., mixtures, thereof. Preferably, the modulator is selected from the group consisting of pentoxifylline, lisofylline, and salts and esters thereof; C-peptides and fragments thereof; combinations of C-peptide or fragment thereof and a source of a pharmaceutically acceptable polyvalent metal cation; complexes comprising a C-peptide or fragment thereof and a polyvalent metal cation; and combinations thereof.

In some embodiments, the modulator is a non-C-peptide compound selected from the group consisting of pentoxifylline, lisofylline, epoxidated arachidonic acids, and salts and esters thereof, and mixtures thereof. Alternatively, the modulator can be a C-peptide compound, complex or mixture selected from the group consisting of: C-peptides and fragments thereof; combinations of C-peptide or fragment thereof and a source of a pharmaceutically acceptable polyvalent metal cation; complexes comprising a C-peptide or fragment thereof and a polyvalent metal cation; and combinations thereof. In one embodiment, the modulator comprises a pharmaceutically acceptable complex comprising a C-peptide and a polyvalent metal cation. The modulator may, in addition or in the alternative, comprise a C-peptide fragment and a polyvalent metal cation. In some embodiments, two or more erythrocyte ATP-release modulators are administered to a subject, e.g., both a C-peptide composition and a non-C-peptide compound.

### C-Peptides

The present technology provides C-peptide/polyvalent metal cation complexes comprising a C-peptide, or a fragment thereof, and a polyvalent metal cation, preferably a divalent or trivalent metal cation. Alternatively, a C-peptide and an suitable polyvalent cation can also be administered separately to a subject, with complex formation taking place in vivo.

As used herein, the term “C-peptide” refers to a polypeptide comprising an amino acid sequence of a C-peptide, preferably a native C-peptide, such as is produced during normal proinsulin processing to form insulin. Preferably, the sequence does not comprise an insulin A-chain or B-chain amino acid sequence, although in some embodiments, about 5 or fewer than 5 residues of one or both of these can be present. Native C-peptides typically are from about 26 to about 32 amino acid residues long. A “native” C-peptide refers to a C-peptide of a proinsulin molecule found in nature. SEQ ID NOs:2-7, 9, and 11-37 present examples of useful native C-peptide amino acid sequences.

C-peptides useful herein can be selected in accordance with the species of the subject to whom it is to be administered, including human or other animal C-peptides. In a preferred embodiment, the C-peptide is human, such as a human C-peptide amino acid sequence shown in SEQ ID NO:2. C-peptide variants as described in PCT Pub. No. WO 2005/039627, Ekberg et al., published May 6, 2005, can also be used in some embodiments.

In various embodiments, the C-peptide of a C-peptide/Cr(III) complex hereof can have an amino acid sequence that is homologous to a C-peptide of the subject to whom the complex is to be administered. A “homologous” amino acid sequence of a C-peptide hereof refers to an amino acid sequence that is at least 80% similar to that of a native C-peptide and that retains the acidic (i.e. Asp and/or Glu) residues of that native C-peptide. In some embodiments, such a homologous amino acid sequence can be at least 80% identical to the native sequence, i.e. while retaining the acidic residues thereof. In various embodiments, the homologous amino acid sequence can be at least or about 85, 90, or 95% similar or identical to the native sequence; in some embodiments, the homologous amino acid sequence can be at least 81, 84, 87, 93, or 96% similar or identical to the native sequence.

The composition and methods of the present technology may comprise a C-peptide fragment. In general, references to “C-peptide” herein are to include C-peptide fragments, which may be used in the compositions and methods of this technology in combination with, or instead of, a C-peptide. As referred to herein, a “fragment” is a peptide comprising amino acid residues that consist of a portion, but not the entirety, of a C-peptide or a homolog thereof, as described above. Thus, in various embodiments, a fragment may comprise less than about 26 to 32 amino acid residues. Fragments may comprise 20 or less, 15 or less, or 10 or less residues. Fragments may comprise 5 or more, 10 or more or 15 or more residues. Examples of fragments include SEQ ID NOs:38-45, set forth in the table, below. Fragments may comprise substitutes of amino acids found in C-peptides. The order of amino acids within fragments may also be altered from those in a C-peptide, such as SEQ ID NO:45. In various embodiments, a fragment comprises a peptide comprising the residue of SEQ ID NO:38.

C-PEPTIDE FRAGMENTS

| SEQ ID NO | TITLE | SEQUENCE |
|---|---|---|
| 38 | C-peptide residues 27-31 | GLU-GLY-SER-LEU-GLN |
| 39 | C-peptide residues 20-31 | SER-LEU-GLN-PRO-LEU-ALA-LEU- |
| | | GLU-GLY-SER-LEU-GLN |
| 40 | C-peptide residues 10-31 | VAL-GLU-LEU-GLY-GLY-GLY-PRO- |
| | | GLY-ALA-GLY-SER-LEU-GLN-PRO- |
| | | LEU-ALA-LEU-GLU-GLY-SER-LEU- |
| | | GLN |
| 41 | C-peptide residues 11-19 | GLU-LEU-GLY-GLY-GLY-PRO-GLY- |
| | | ALA-GLY |
| 42 | C-peptide residues 1-13 | GLU-ALA-GLU-ASP-LEU-GLN-VAL- |
| | | GLY-GLN-VAL-GLU-LEU-GLY |
| 43 | C-peptide residues 27-31 | ALA-GLY-SER-LEU-GLN |
| | (E27A) | |
| 44 | C-peptide residues 27-31 | ASP-GLY-SER-LEU-GLN |
| | (E27D) | |
| 45 | C-peptide residues 27-31 | SER-GLN-LEU-GLU-GLY |
| | (scrambled) | |

C-peptides and fragments can be isolated from natural sources or produced by recombinant technology. Recombinant C-peptides and fragments can be prepared by any suitable method including those known in the art, based on use of an expressible nucleic acid encoding, e.g., a native C-peptide amino acid sequence, such as by use of a nucleotide sequence of SEQ ID NOs:1, 8, or 10 hereof. In some embodiments, concatamers of such peptides with one another or fusion peptides with a further polypeptide can be designed to provide enzymatic or chemical (e.g., acid hydrolysis) cleavage sites between adjacent peptide sequences in an expressed molecule in order to permit release of C-peptides for use herein. After production, C-peptides and fragments can be recovered and, if desired, further purified to remove most extraneous contaminants, using any of the various techniques known in the art, e.g., column chromatography, immunoseparation, gel electrophoresis, and the like.

### C-Peptide Complexes

In various embodiments, the C-peptide or fragment is combined in vitro or in vivo with a pharmaceutically acceptable polyvalent metal cation. In some embodiments, polyvalent cations are divalent or trivalent. In various embodiments, the metal utilized for the polyvalent cation can be any pharmaceutically acceptable alkaline earth metal, transition metal, lanthanide, or Al, Ga, In, or Bi; or combination thereof. Such cations include: divalent Mg, Ca, Sr, Ba, Ge, or Sn cations; trivalent Al, Ga, In, or Bi cations; polyvalent (e.g., di- or tri-valent) transition metal cations; and polyvalent (e.g., di- or tri-valent) lanthanide cations, e.g., La, Ce, Pr, Nd, Pm, Sm, Eu, Gd, Tb, Dy, Ho, Er, Tm, Yb, or Lu cations; and combinations thereof. The cation can also be a polyvalent transition metal cation or a combination thereof. Transition metal cations include a polyvalent V, Cr, Mn, Fe, Co, Ni, Cu, Zn, Mo, Ag, Pt, and Au cations, and combinations thereof. Metal complexes can also include a combination of polyvalent metal cations or one or more monovalent metal cations, e.g., alkali metal cations. In some preferred embodiments, a polyvalent Cr, Mn, Fe, or Zn cation, or a combination thereof, can be used; or Cr(III), or Fe(II) or Zn(II); or Cr(III); or Zn(II). The c-peptide/polyvalent metal cation complexes may comprise from about 10 to about 67 mole percent polyvalent metal cation, based on the total moles of ions present in the complex.

Complexes can comprise, in addition to the metal cation(s) and C-peptide or fragment, one or more further pharmaceutically acceptable, mono- or di-valent anions, or electron donors. Such anions include halide, oxyacid, and other anions, including those commonly found in commercially available Cr(III) salts, such as esters, halides (e.g., chloride or bromide), and physiologically acceptable acids, including carboxylic acids (e.g., polycarboxylic acids), amino acids, sulfoxy acids (e.g., sulfate, bisulfate, sulfonate), phosphoxy acids (e.g., phosphate, biphosphate, phosphonate, biphosphonate), carbonate, bicarbonate, nitrate, aromatic acids, nucleoside phosphates, and their esters. Examples of chromium complexes and salts useful herein include: chromium picolinate, chromium citrate, chromium chloride, chromium aspartate, Cr-ATP complexes (e.g., Cr-ATP-Cys₂), Cr-ADP complexes, chromium trinicotinate, chromium dinicotinate chloride, Glucose Tolerance Factor (GTF), and the like. At physiological pH, GTF is reported to comprise Cr(III) complexed with one O-glutathionyl ligand and two O-nicotinyl ligands. Such electron pair donors and anions are also useful in forming mixed complexes containing Cr(III) and C-peptide. In some embodiments, the anions or electron donor(s) present in such metal compounds can be selected for use as a further component in a C-peptide complex hereof.

The present technology also provides processes for preparing a C-peptide (fragment)/polyvalent metal cation complex, comprising:

(A) providing an isolated or recombinant C-peptide or fragment and a source of a pharmaceutically acceptable polyvalent metal cation; and

(B) contacting the peptide and cation source under conditions in which the cation can attach to acidic residues of the peptide, thereby forming a complex. Kits are also provided for the preparation of a C-peptide/polyvalent metal cation complex, the kit comprising a frozen or lyophilized C-peptide or fragment, and a source of a pharmaceutically acceptable polyvalent metal cation, with instructions for preparing a C-peptide/polyvalent metal cation complex therefrom, optionally further comprising instructions for making a pharmaceutical formulation containing the complex, and optionally further comprising instructions for administering the formulation to a human or animal subject.

### Antibodies

In some embodiments hereof, antibodies are provided that have binding specificity for a C-peptide/polyvalent metal cation complex. In some embodiments, an anti-C-peptide/polyvalent metal cation complex antibody can have selectivity for, or greater affinity for, the complex as versus the same C-peptide or fragment not complexed with a polyvalent metal cation. In some embodiments hereof, antibodies are provided that are anti-idiotypic antibodies to such an anti-C-peptide/polyvalent metal cation complex antibody. These anti-complex and anti-idiotypic antibodies can be used for diagnostic, screening, and/or therapeutic purposes.

### Compositions

The present technology provides compositions comprising a C-peptide/polyvalent metal cation complex or other erythrocyte ATP-release modulator and a pharmaceutically-acceptable carrier. The compositions of this invention are preferably provided in unit dosage form. As used herein, a “unit dosage form” is a composition of this invention containing an amount of an erythrocyte ATP-release modulator that is suitable for administration to a human or lower animal subject, in a single dose, according to good medical practice. In general, the amount of erythrocyte ATP-release modulator in a unit dose composition of this invention is the safe and effective amount of said erythrocyte ATP-release modulator to be administered on a daily basis, such as is discussed further below, divided by the number of doses of said compound to be given in a day. The “number of doses” for a given erythrocyte ATP-release modulator is the number of doses necessary to maintain an effective concentration of the compound at the site(s) at which the compound is to have a therapeutic effect. The safe and effective amount and number of doses will vary according to the erythrocyte ATP-release modulator and its pharmacokinetic characteristics, the disorder to be treated, and the route of administration. A “safe and effective” amount of an erythrocyte ATP-release modulator is an amount that is sufficient to have the desired therapeutic effect in the human or other animal subject, without undue adverse side effects (such as toxicity, irritation, or allergic response), commensurate with a reasonable benefit/risk ratio when used in the manner of this invention. The specific safe and effective amount of the erythrocyte ATP-release modulator will vary with such factors as the particular condition being treated, the physical condition of the patient, the nature of concurrent therapy (if any), the specific erythrocyte ATP-release modulator used, the specific route of administration and dosage form, the carrier employed, and the desired dosage regimen.

The compositions of this technology can be in any suitable dosage form, such as for enteral, parenteral, or topical administration. The specific carrier may comprise one or more materials, and may be adapted for the intended route of administration for the composition. Such carrier materials include diluents, lubricants, binders, solvents, dissolution promoters, penetration enhancers, buffers, preservatives, flavorants, fragrances, sweeteners, and colorants. In particular, for example, transdermal formulations can comprise skin-enhancing agent(s), enteral formulations for oral administration can comprise a flavoring, viscosity modifier, or mouth-feel-improving agent, and formulations for nasal administration can comprise a fragrance.

Topical compositions may comprise a penetration enhancer. For example, in some embodiments, transdermal or transmucosal delivery can involve co-application of, e.g.: iontophoretic or electrophoretic agents or conditions, or surfactants or solubilizers, e.g., dimethylsulfoxide (DMSO). Gel formulations therefor can comprise any of the various gel bases known in the art as suitable for topical gel preparations. In some embodiments, a gel formulation can comprise as a gelling agent, a polysaccharide or derivative, e.g., C1-C4 alkyl ether or hydroxyalkyl ether thereof, a polyglycuronate, and the like. In some embodiments, this can be an adhesive gel, such as that described in R. D'Souza et al., Nasal insulin gel as an altenative to parenteral insulin: Formulation, preclinical, and clinical studies, AAPS PharmSciTech 6(2):E184-89 (Sep. 30, 2005) (using an adhesive gel of carbopol 934P and HPMC).

In some embodiments, a parenteral formulation can comprise an additive that slows the rate of release of the active ingredient(s). In some embodiments, such an additive can comprise a protamine, e.g., any of: human protamine 1 (PRM1; Genbank NP_{—}002752.1.gi:4506109), human protamine 2 (PRM2; NP_{—}002753.2.gi:68989267), human protamine 3 (PRM3; NP_{—}067070.1.gi:10864057), a salmine (from Oncorhynchus sp.; e.g., Genbank P69014.gi:60392939), an iridine (e.g., Genbank PO2328.gi:131042), a clupeine (from Clupea sp.; e.g., P69011.gi:59800150), others, and combinations thereof. Protamines or other release-delaying agents can likewise be used in non-parenteral formulations. Many examples of other useful additives are well known in the art and can be used herein, e.g., those described in U.S. Pat. No. 6,551,992, DeFelippis et al., issued Apr. 22, 2003.

Enteral formulations can comprise, e.g., a bile acid or salt or ester, such as a cholate, examples of which include glycocholate, taurocholate, deoxycholates, C1-C4 alkyl-cholates; a protease inhibitor, such as a trypsin inhibitor; or a surface active or delivery-enhancing agent such as sodium N-[8-(2-hydroxybenzoyl)amino]caprylate SNAC), described in M. Kidron et al., A novel per-oral insulin formulation: proof of concept study in non-diabetic subjects, Diabetic Med. 21(4):354-57 (April 2004) (e-Publ. doi:10.1111/j.1464-5491.2004.01160.x) (reporting effective serum delivery of insulin from enteral administration of an encapsulated combination comprising the peptide and SNAC).

Powders for inhalation can be based on dried formulations of the pharmaceutical agents of the composition, or by application of the agent(s) to a core particle. Some useful examples of such formulations are described, e.g., in: R. Vanbever et al., Sustained release of insulin from insoluble inhaled particles, Drug Devel. Res. 48(4):178-85 (1999) (e-Publ. 11 Feb. 2000) (forming drug particles by spray drying a mixture of insulin with either protamine or zinc chloride onto lactose-albumin-dipalmitoylphosphatidylcholine core particles); U.S. Pat. No. 6,984,404, Talton et al., issued Jan. 10, 2006, for Methods for preparing coated drug particles and pharmaceutical formulations thereof; and U.S. Pub. No. 2006/0177417, Musso et al., published Aug. 10, 2006, for Pharmaceutical formulations for sustained drug delivery.

Particles can typically have a diameter of about 1 nm to about 50 μm. In some embodiments, core particles for use therein can be formed from, e.g.: (1) saccharides, polysaccharides, and their derivatives, e.g., celluloses, starches, polyglycuronates such as alginates, C1-C4 alkyl or hydroxyalkyl ethers thereof, oxyacid esters therewith, and the like; (2) biocompatible polyesters, e.g., poly(C2-C8)hydroxyalkanoates, such as PLA, PGA, PLGA, PHB, PCL, and the like; (3) polyacrylates; (4) silica; or (5) mixtures thereof with one another and/or with further component(s) such as polypeptides, surfactants, lipids, biocompatible adhesives, and/or other components.

In some embodiments, a long-term drug delivery mode can be utilized, as by implantation of a sustained release drug delivery composition/depot or device. Some useful examples of these are described in: U.S. Pat. No. 4,774,091, Yamahira et al., Sep. 27, 1988, for Long-term sustained-release preparation; P. Y. Wang, in Biomaterials 12(1):57-62 (January 1991) and in Int. J. Pharm, 54:223-30 (1989) (using a long-chain fatty acid or triglyceride as excipient for sustained release insulin delivery); U.S. Pat. No. 5,062,841, Siegel, issued Nov. 5, 1991, for Implantable, self-regulating mechanochemical insulin pump; M. Ferrari 7 J. Liu, in Mech. Eng. (December 2001); S. S. Iyer et al., in Biopharm. & Drug Dispos. 27(4):157-70 (e-Pub. Jan. 17, 2006); and U.S. Pat. No. 6,656,508, Goldenberg et al., issued Dec. 2, 2003.

In various embodiments, a composition comprising a C-peptide or C-peptide complex is of a type used for the administration of insulin. Examples of useful methods and compositions, already described as useful for insulin delivery, can be found, e.g., in: U.S. Pat. No. 5,884,620, Gonda et al., issued Mar. 23, 1999; U.S. Patent Publication 2004/0112376, Chaiken et al., published Jun. 17, 2004; U.S. Patent Publication 2005/0220722, Landschulz, published Oct. 6, 2005; U.S. Patent Publication 2006/0067891, Modi, published Mar. 30, 2006; U.S. Pat. No. 7,070,799, Modi, issued Jul. 4, 2006; PCT Pub. No. WO 2004/016243, Modi, published Feb. 26, 2004; PCT Pub. No. WO 01/87268, Modi, published Nov. 22, 2001; PCT Pub. No. WO 01/66085, Modi, published Sep. 13, 2001; and U.S. Pat. No. 5,449,670, Skinner et al., issued Sep. 12, 1995.

In some embodiments, an erythrocyte ATP-release modulator can be further combined with other bioactive agents. Such bioactive agents can be, for example, pharmaceutical, nutraceutical, or nutritive agent(s). In some embodiments, a further pharmaceutical agent can be included, such as a small molecular or biomolecular pharmaceutical.

In various embodiments, the compositions comprise a glucose metabolism modulator. Glucose metabolism modulators useful herein include insulin, hypoglycemic agents, and mixtures thereof. As referred to herein, “insulin” includes native insulin as well as naturally-occurring and synthetic analogs of insulin as are known in the art, some examples of which include: insulin aspart (available as NovoLOG from Novo Nordisk A/S, Copenhagen, DK); insulin detemir (available as LEVEMIR from Novo Nordisk); insulin glargine (available as LANTUS from sanofi-aventis US LLC, Bridgewater, N.J., USA); insulin glulisine (available as APIDRA from sanofi-aventis); and insulin lispro (available as HUMALOG from Eli Lilly & Co., Indianapolis, Ind., USA). Hypoglycemic agents include oral agents such as tolbutaminde, chlorpropamide, tolazamide, acetohexamide, glyburide, glipizide, gliclazide, and mixtures thereof.

Compositions can optimally comprise a C-peptide/polyvalent metal cation complex and an agent that promotes stability of the complex. Examples of such stability agents include pharmaceutically acceptable: water soluble anionic polymers, e.g., containing carboxyl or sulfonyl groups; chelants, e.g., carboxylate chelants such as NTA, EDTA, EGTA, EDTP, or EDDS; and polyols, e.g., saccharides. In some embodiments, the moles of complex-stabilizing agent(s) can be about or less than the number of moles of C-peptide present in the composition. In some embodiments, the composition can be essentially free of alkali metal cations, e.g., lithium, sodium, and potassium ions; in some embodiments, the composition can contain such cations in an amount that is about 30 mol. % or less of all cations present in the composition, or that is about 25, 20, 15, or 10 mol. % or less.

### Methods of Treatment

The present technology provides methods for modulating erythrocyte ATP-release in human or other animal subjects. Methods comprise administering to a human or animal subject a safe and effective amount of an erythrocyte ATP-release modulator, preferably by administering a composition comprising a safe and effective amount of an erythrocyte ATP-release modulator and a pharmaceutically acceptable carrier. Such methods include the treatment or prevention of disorders associated with reduced ATP release by erythrocytes, i.e., disorders in which erythrocytes exhibit lower than normal ATP release under physiologic conditions, or in which symptomatic or physiological improvement is effected by increase in ATP release by erythrocytes.

Methods include those for modulating glucose metabolism, and methods for promoting vasodilation in human or other animal subjects. In some embodiments, an erythrocyte ATP-release modulators can be used to treat a vascular condition, such as, but not limited to: hypertension; gestational hypertension; peripheral vascular diseases; chronic venous insufficiency; Raynaud's disease; such conditions in other disorders, e.g., Raynaud's involvement in scleroderma, lupus, Sjögren's syndrome, or rheumatoid arthritis; and vascular aspects of cardiac care, of recovery following heart failure, of stroke, of recovery following stroke, or of erectile dysfunction. In some embodiments, an erythrocyte ATP-release modulator can be used to treat a glucose processing or metabolism disorder, such as, but not limited to: diabetes mellitus type 1 or type 2, gestational diabetes, hyperglycemia, or metabolic syndrome. An erythrocyte ATP-release modulator may also be used to treat other disorders, such as those associated with RBC membranes described above, e.g., malaria, chronic fatigue syndrome, and obesity. In various embodiments, methods comprise administering a safe and effective amount of an active selected from the group consisting of pentoxifylline, lisofylline, epoxidated arachidonic acids, and salts and esters thereof; C-peptide or fragment; mixtures of C-peptide or fragment and a source of a pharmaceutically acceptable polyvalent metal cation; complexes comprising a C-peptide or fragment and a polyvalent metal cation; and mixtures thereof.

For example, the present technology provides methods for treating vascular conditions, such as promoting vasodilation in a human or other animal subject in need thereof, comprising administering to the subject a composition comprising a therapeutically effective dose of an erythrocyte ATP-release modulator. Vascular conditions can be of non-diabetic etiology, or a diabetes-based vascular pathology or complication.

Methods of the present technology comprise administering of an erythrocyte ATP-release modulator by any suitable route of administration, including enteral, parenteral or topical administration. Among parenteral modes, intravenous, subcutaneous (including subdermal), intramuscular, and intraperitoneal routes can be employed. These can be effected by, e.g., perfusion or injection. Injection can be accomplished by use of a hypodermic needle or a fluid microjet. Suspension, solution, and emulsion formulations are all considered useful in various embodiments for parenteral administration. Among topical modes, transdermal, transpulmonary, and transmucosal routes are considered desirable. These can be effected by, e.g., direct surface application of a gel or spray, or inhalation of solid or liquid particles/droplets. In various embodiments, transdermal administration can be performed using, e.g., an ointment or gel or an adhesive article, such as a film, patch, or strip. Transpulmonary administration can take place by, e.g., transbronchial or transalveolar absorption of an inhaled powder, spray, or mist. Transmucosal administration can be performed by application of a liquid, spray, or gel to the mucous membrane for, e.g., transbuccal or transnasal absorption. Enteral routes commonly involve administration of an encapsulated formulation for dissolution in the small intestine.

In various embodiments, the present technology provides methods for promoting glucose clearance or vasodilation in a human or animal subject, comprising administering to the subject a safe and effective amount of a pharmaceutically acceptable C-peptide/metal cation complex in which the metal cation comprises a pharmaceutically acceptable M(II) or M(III) cation or other erythrocyte ATP-release modulator. In various embodiments, such methods for promoting glucose metabolism are performed in subjects having diabetes mellitus type 1, diabetes mellitus type 2, gestational diabetes, or metabolic syndrome. The method may be a prophylactic treatment for a subject identified as being at risk for developing a disorder of glucose processing, or a palliative treatment for a subject having a glucose processing disorder.

The present technology also provides regimens for treating diabetes mellitus in a human or other animal subject comprising administering to the subject a glucose metabolism modulator and erythrocyte ATP-release modulator, wherein said erythrocyte ATP-release modulator is effective to reduce the level of the glucose metabolism modulator needed to effect glucose control in the subject, extend the duration of efficacy of the glucose metabolism modulator in the subject, or both. The glucose metabolism modulator may be, for example, insulin or a hypoglycemic agent. In various embodiments, the erythrocyte ATP-release modulator and glucose metabolism modulator are administered at “synergistic” levels. In such methods, the therapeutic effect of administering of the combination of the erythrocyte ATP-release modulator and glucose metabolism modulator is greater than the additive effect of administering erythrocyte ATP-release modulator and glucose metabolism modulator individually. Such effects include one or more of increasing the effect of the glucose metabolism modulator, increasing the duration of the effect of the glucose metabolism modulator, and making glucose metabolism modulator effective at dosage levels that would otherwise be ineffective.

In various methods, a C-peptide/polyvalent metal cation complex may be formed in vivo, consequent to separate administration of a C-peptide and a polyvalent metal cation. Thus, methods are provided comprising administering a first composition comprising a C-peptide and administering a second composition comprising cation-releasable source of the metal cation. The two administering steps may be substantially concurrent, or may be performed in either order separated by minutes, hours or days. Optionally, a glucose metabolism modulator may also be administered. In one embodiment, a composition is administered comprising a glucose metabolism modulator and a cation-releasable material.

### Dosages

The specific dosage level of erythrocyte ATP-release modulator administered will depend on a variety of factors according to standard medical practice. Specific dosages may depend on such factors as the particular erythrocyte ATP-release modulator administered, its pharmacokinetic characteristics, the disorder to be treated, and the route of administration.

In various embodiments, serum concentrations of C-peptide or fragment in humans can range from about 5 pmol/L to about 5 nmol/L. In some embodiments in which C-peptide, fragment, or a C-peptide complex is to be employed for treating a glucose-processing disorder, the amount of C-peptide selected for use in a given dose can be varied, based on factors such as: the endogenous level of C-peptide in the subject's serum; whether or not the administration is to be coordinated with a fasting or non-fasting state; dietary considerations, e.g., administration for a high carbohydrate meal; the mode of administration and its rate of serum delivery of active ingredient; and so forth. For example, subcutaneous administration can utilize a higher than physiological dose, e.g., 75-100 nmol, in order to provide a serum concentration within the range of, e.g., about 10-200 μmol/L. Similar considerations apply for selection of a C-peptide or -complex dose to be employed for promoting vasodilation.

In various embodiments, the dose can be any that provides a serum concentration of C-peptide or fragment that is approximately equal to that of the subject's serum insulin concentration, on a mole-to-mole basis. In some embodiments, the C-peptide, fragment, or C-peptide complex dose can be any that provides a serum concentration of C-peptide or fragment that is from about 10 μmol/L to about 4 nmol/L, from about 20 μmol/L to about 2 nmol/L, or from about 50 μmol/L to about 1 nmol/L; or that is about or at least 100 or 500 μmol/L; and that is up to or about 5 nmol/L. In some embodiments, the serum C-peptide or fragment concentration can be from about 10 to about 500 μmol/L, about 10 to about 200 μmol/L, about 50 to about 180 μmol/L, about 60 to about 150 μmol/L, or about 50 to about 100 μmol/L; such concentrations are considered particularly useful for, e.g., intravenous, transmucosal, and enteral formulations. In some embodiments, an administered C-peptide or fragment dose can be from about 1 to about 20 nmol/L, from about 2 to about 20 nmol/L, about 5 to about 15 nmol/L, or about 10 nmol/L; such concentrations are considered particularly useful for, e.g., subcutaneous, transdermal, and lipid depot modes of administration. In some embodiments, the target serum concentration for C-peptide, fragment, or C-peptide complex can be at least or about 5, 10, 20, 50, 60, 70, 80, 100, 120, 150, 180, or 200 μmol/L; and/or can be up to or about 4,000, 3,000, 2,000, 1,000, 500, 400, 200, 180, 150, 120, 100, 80, 70, 60, or 50 μmol/L. In some cases, reported serum C-peptide or fragment concentration can be found that are about 1/10 to about 1/30 that of the serum insulin concentration. Thus, in various embodiments, the dose can be any that provides a serum concentration of C-peptide or fragment that is approximately 1/10 to 1/30 that of the subject's serum insulin concentration, on a mole-to-mole basis; and such cases, a serum concentration or administered dose of C-peptide, fragment, or C-peptide complex hereof can be about 1/10 to about 1/30 of those described hereinabove. Similarly, in some embodiments of compositions hereof containing a C-peptide, fragment, or C-peptide complex with insulin, the mole-to-mole ratio of C-peptide (or fragment) to insulin can be from about 0.03:1 to about 1:1, or more.

Examples of useful dosages for pentoxifylline and lisofylline can be from about 0.01 to about 6 mg/kg, with a maximum of about 400-500 mg per human subject. In some embodiments pentoxifylline and lisofylline can be administered is dosages of about 0.1 to about 4 mg/kg, or about 0.5 to about 3 mg/kg. Similar dose ranges can be useful for epoxyeicosatrienoic acids.

Any of various dosage regimens can be used to administer a composition according to the present invention. For example, any dosage regimen known useful for administration of an insulin formulation can be used for a comparable formulation of an erythrocyte ATP-release modulator hereof.

Diagnostic methods for assessing the level of ATP release from erythrocytes are disclosed in PCT Pub. No. WO 2008/118390, Spence, published Oct. 2, 2008, incorporated by reference herein. Such methods may be used, for example, to identify subjects at risk of a glucose metabolism disorder, vascular disorder, or disorder associated with reduced erythrocyte ATP release for treatment by methods of the present technology. Thus, in various embodiments, methods are provided wherein the health of a subject is assessed comprising assaying erythrocytes of the subject for their level of ATP release upon physical deformation and comparing that level to a normal range of ATP release and, if the level of ATP release is significantly reduced compared to the normal range, administering to the subject a composition of the present technology. In some embodiments thereof in which a significant level of RBC ATP release can be detected without deformation, deformation is optional. Such diagnostic methods may also be used to monitor the status of the treatment methods of the present technology, for example to assess the efficacy of treatment and the proper dosage level of the C-peptide/polyvalent metal cation complex or other erythrocyte ATP-release modulators of the present technology.

### EXAMPLES

### Materials & Methods

Genetic techniques can be performed according to commonly known methods of nucleic acid manipulation, such as those described in: FM. Ausubel et al. (eds.), Current Protocols in Molecular Biology (2006) (Wiley Interscience, NY); F. M. Ausubel et al., Short Protocols in Molecular Biology (2002) (5^{th} ed.; John Wiley & Sons); Sambrook & Russell, Molecular Cloning: A Laboratory Manual (2001) (Cold Spring Harbor Lab., NY); W Ream & K Field, Molecular Biology Techniques: An Intensive Laboratory Course (1998) (Academic Press); C. R. Newton & A. Graham, “PCR,” in series Introduction to Biotechniques (1997) (2^{nd} ed.; Springer Verlag); and Berger & Kimmel (eds.), “Guide to Molecular Cloning Techniques,” in series Methods in Enzymology 152 (1987) (Academic Press, San Diego, Calif.).

Pharmaceutical formulations for administration can be prepared by any useful method known in the art, such as those described in: A. R. Gennaro et al., Remington: The Science and Practice of Pharmacy (2005) (21st ed.; Lippincott Williams & Wilkins, Phil., PA) (Univ. Sci. in Phil., PA); R. C. Rowe et al., Handbook of Pharmaceutical Excipients (2005) (APHA Publications, Washington, D.C.); L. Brunton et al., Goodman & Gilman's The Pharmacological Basis of Therapeutics (2005) (11^{th} ed.; McGraw-Hill Professional, New York, N.Y.); and S. K. Niazi, Handbook of Pharmaceutical Manufacturing Formulations (2004) (Informa Healthcare, London, UK) (esp. vol. 2).

Preparation of RBCs. Rabbits (New Zealand whites, males, 2.0-2.5 kg) were anesthetized with ketamine (8 ml/kg, i.m.) and xylazine (1 mg/kg, i.m.) followed by pentobarbital sodium (15 mg/kg i.v.). A cannula was placed in the trachea and the animals were ventilated with room air. A catheter was placed into a carotid artery for administration of heparin and for phlebotomy. After heparin (500 units, i.v.), the animals were exsanguinated. Human blood was obtained by venipuncture without the use of a tourniquet (antecubital fossa) and collected into a heparinized syringe. Blood was centrifuged at 500×g at 4 C for 10 min. The plasma and buffy coat were discarded. The RBCs were resuspended and washed three times in a physiological salt solution [PSS, containing in mM: 4.7 KCl, 2.0 CaCl₂, 140.5 NaCl 12 MgSO₄, 21.0 tris(hydroxymethyl)aminomethane, 11.1 dextrose with 5% bovine serum albumin (final pH 7.4)]. Cells were prepared on the day of use and experiments were finished within 8 hours of removal from the animal or human subjects. All procedures were approved by the Animal Investigation Committee or the Human Investigation Committee at Wayne State University.

Measurement of ATP. Human C-peptide (American Peptide Co., Sunnyvale, Calif.), 0.25 mg (MW=3020 g/mol), was dissolved in 100 mL of purified water (18.2 megaohm) to yield a concentration of 83 μM. Next, an appropriate volume of this C-peptide solution was added to 10 mL of a 7% solution of RBCs to create a solution containing the C-peptide at concentrations in the 1-5 nM range. The RBC-peptide solution was immediately loaded into a 500 μL syringe and placed on a syringe pump; the other syringe contained a solution of luciferin/luciferase (Sigma, FLE-50 with 2 mg of added luciferin to improve sensitivity). Both solutions were pumped simultaneously at a rate of 6.70 μL/min through portions of fused-silica microbore tubing (50 μm i.d., 365 μm o.d., Polymicro Technologies, Phoenix, Ariz.) to a mixing tee. The resulting chemluminescence reaction flowed through a final potion of fused-silica microbore tubing that was placed over a photomultiplier tube, where the emission was detected. The resultant current was measured as a potential by a data acquisition board operated by a program written with the LabView software package (National Instruments, Austin, Tex.).

To ensure that the resulting increase in ATP release was due to C-peptide interacting with the RBCs and not cell lysis, the RBC solutions were measured under non-flow conditions using a luminometer with 200 μL of the RBC solution and 200 μL of the luciferin/luciferase solution. No detectable signals were obtained. To ensure that lysis was not occurring in the tubing, a solution of 0.01 M glybenclamide was prepared by adding 49 mg of glybenclamide (Sigma) to 2 mL of a 0.1 M solution of sodium hydroxide. To this, 7.94 mL of a dextrose solution (1 g dextrose in 20 mL of purified water) was added. The mixture was heated carefully to 52° C. until all of the glybenclamide was dissolved. Once the solute was completely dissolved, 1 mL of this solution was added to 9 mL of PSS, resulting in a solution with a concentration 0.001 M. From this diluted solution, 2.5 mL were added to 2.5 mL of 7% hematocrit RBC solution, resulting in a 3.5% hematocrit solution of RBCs. This solution was allowed to incubate for 15 minutes. As a comparison, 2.5 mL of wash buffer without glybenclamide was added to 2.5 mL of 7% hematocrit RBCs. After 15 minutes, the RBC solutions were assayed.

Introduction. C-peptide may be able to mediate the production of endothelium-derived NO via its ability to increase the levels of ATP released from erythrocytes that are subjected to mechanical deformation. Here, studies are performed in which RBCs are pumped through microbore tubing having diameters that approximate those of resistance vessels in vivo. Upon deformation in the tubing, the RBCs release ATP that is measured using a well-established chemiluminescence assay for ATP. The concentrations of RBC-derived ATP are measured in the presence and absence of synthetic C-peptide. Mass spectrometric data unexpectedly reveals that binding of the C-peptide to a polyvalent metal cation, here using chromium (III), is necessary for extended activity of the peptide.

### Example 1

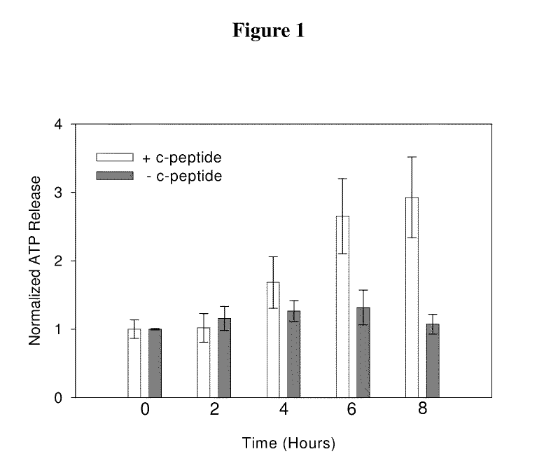

C-Peptide-Induced Release of ATP. RBCs obtained from rabbits are pumped through microbore tubing having an inside diameter of 50 m and the resultant ATP released by the cells upon deformation in the tubing is measured. See, J. S. Carroll et al., in Mol. Biosys. 2:305-311 (2006); R. Sprung et al., in Anal. Chem. 74:2274-2278 (2002). Another aliquot from the same RBC sample is incubated in 1 nM C-peptide and the resultant ATP release measured every 2 h for a period up to 6 h. As shown in FIG. 1, which contains normalized values of ATP released from the RBCs of n=11 rabbits, the C-peptide has the ability to increase the deformation-induced release of ATP from the RBCs. The data shown are normalized values from the RBCs of n=12 rabbits incubated in the presence and absence of 1 nM c-peptide. As shown, the ATP release (determined by a chemiluminescence assay) from those cells incubated in the c-peptide increased approximately 2.9 times over a period of 8 h. RBCs in the absence of the c-peptide demonstrated to statistically significant change in their ability to release ATP. Error bars are ±SEM. The increase seen over the 6 h period is nearly three times that of the RBCs incubated with a control (buffer without C-peptide). In addition, the increase in the ATP release can be inhibited when the RBCs are incubated in glybenclamide, a substance known to inhibit ATP release from RBCs. This inhibition demonstrates that the increase in measured extracellular ATP is not due to cell lysis. If cell lysis were occurring, the glybenclamide would have no affect on the measured ATP as it would be present in extracellular form whether or not glybenclamide was introduced to the RBCs.

### Example 2

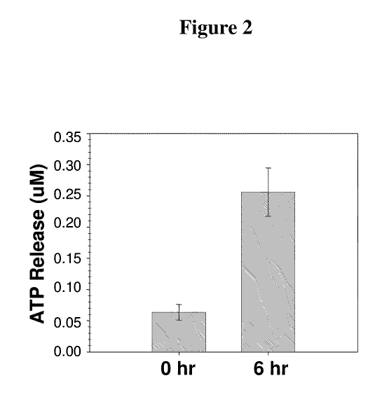

Restoration of ATP Release from the RBCs of Patients with Diabetes. Recently, it has been reported that RBCs obtained from the whole blood of patients with Type II diabetes mellitus release approximately 50% of the ATP released from the RBCs of healthy control patients. Thus, RBCs of diabetic patients may have released less ATP due to oxidative stress within the RBCs, leading to a less deformable cell. A decrease in RBC deformability is a recognized trait of the RBCs obtained from patients with diabetes. See, L. O. Simpson, in Nephron 39:344-51 (1985); R. S. Schwartz et al., in Diabetes 40:701-712 (1991). The ability of C-peptide to restore ATP release in diabetic RBCs is assayed. As shown in FIG. 2, C-peptide administration is now been found to have the ability to increase the ATP release from the RBCs of patients with type II diabetes (n=7). Moreover, this effect is substantial in that such administration has the ability to restore these release levels to a value that is statistically equivalent to that of healthy, non-diabetic control patients.

### Example 3

Mass Spectrometric Analysis of Metal-Peptide Binding. Additional results from repeats of Experiments 1 and 2 initially and unexpectedly failed to confirm the ATP-release modulating effect of C-peptide. Metaanalysis of the collective data surprisingly revealed that a C-peptide preparation would generally lose bioactivity about 24-36 h after preparation in water. Analysis of the C-peptide using electrospray ionization mass spectrometry indicates that the peptide is not undergoing any type of degradation or cleavage, even after remaining in solution for periods >30 days. Thus, alternative postulated causes are tested, including that a covalent modification of the peptide, e.g., induction or lysis of a side-chain-to-side-chain bond or of a moiety covalently attached to an amino acid residue, might be involved, or that non-covalent interaction with another chemical species, e.g., a metal ion, might be involved in this effect.

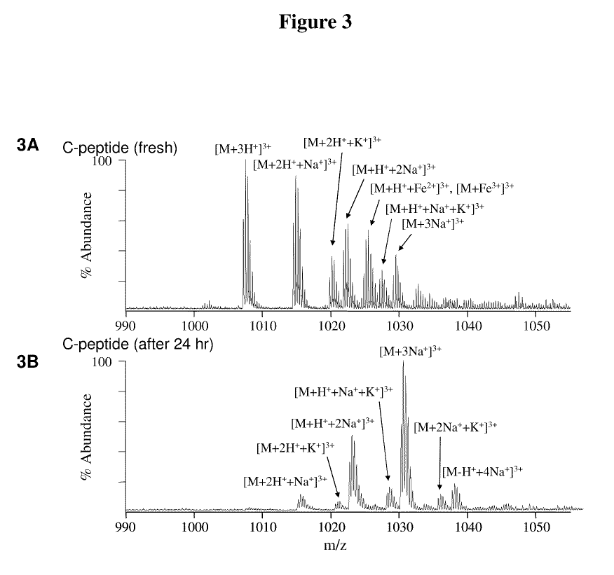

The data in FIG. 3 reveal some information about the possible loss of activity of the C-peptide after preparation in the aqueous solvent. Specifically, the mass spectrum shown in FIG. 3a is that of peptide prepared in water and analyzed within 0.5 h of preparation. In 3a, the [M+3H]³⁺ peak is present as are other forms of the peptide with sodium atoms, potassium atoms, or a combination thereof. Interestingly, there is also a peak that corresponds to binding to an iron atom [M+H⁺+Fe²⁺]³⁺. The presence of this Fe(II) adduct to the C-peptide is not present 24 h after preparation. These data provide evidence suggesting that the activity of the peptide involved binding to metal cation(s).

### Example 4

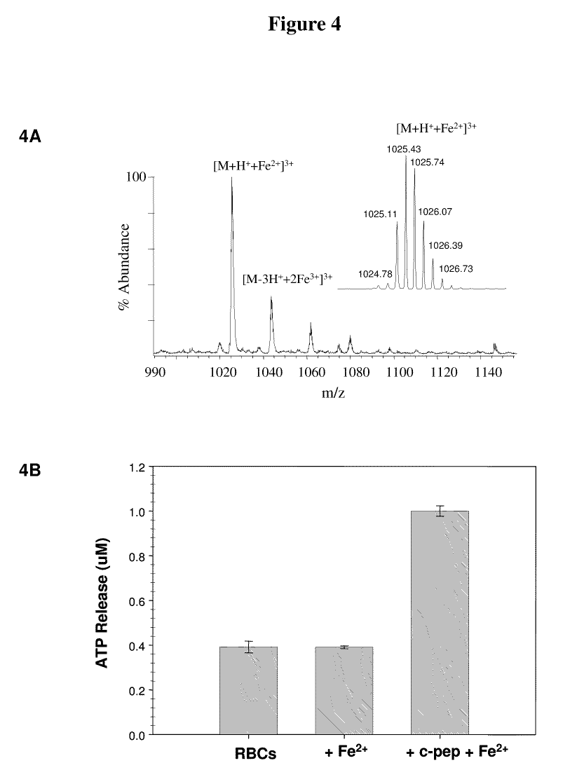

Metal-Induced Activity of C-Peptide. Based on the data shown in FIG. 3, which demonstrates the ability of the C-peptide to bind to Fe(II), RBCs are incubated in solutions containing Fe(II) and their subsequent ability to release ATP upon being subjected to deformation is determined. The data in FIG. 4 is consistent with the data shown in FIGS. 1 and 2; namely, that the activity of the C-peptide is dependent upon its ability to bind to the metal ion. Specifically, RBCs are incubated in C-peptide that has been kept at 4° C. for >30 days; therefore, this solution of C-peptide no longer has the ability to induce ATP release from deformed RBCs. This same inactive C-peptide solution is then combined with an Fe(II) source such that the concentrations of both Fe(II) and C-peptide are 1 nM. This solution containing C-peptide and Fe(II) is then applied to the RBCs and, after 6 h, the RBC-derived ATP is measured. The results in FIG. 4 clearly demonstrate that the activity of the C-peptide can be restored when bound to the Fe(II) metal ion. As a control, the RBCs are incubated with the metal ion in the absence of the peptide and it is found that the solution of metal ion alone does not result in an increase in RBC-derived ATP.

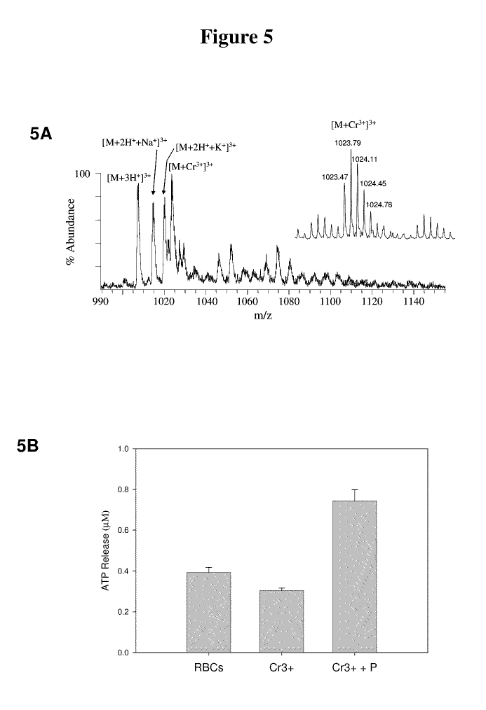

Although the Fe(II)-bound C-peptide has the ability to increase ATP-release from deformed RBCs, its activity also appears somewhat limited. Specifically, while the addition of Fe(II) to inactive C-peptide is able to restore the peptide's activity, it too decreases after 24 h. Moreover, it is found that, beyond 48 h, the activity of the Fe(II)-bound C-peptide generally shows no statistical difference from that of C-peptide alone. Mass spectrometric examination of the Fe(II)-C-peptide adduct, shown in FIG. 5, was found to help explain this observation. The unexpected result is that the population of Fe(II)-C-peptide adduct begins to diminish within 24 h after the addition of an Fe(II) source, and Fe(II) is then replaced by either sodium or potassium, or both, cations in the C-peptide complex.

### Example 5

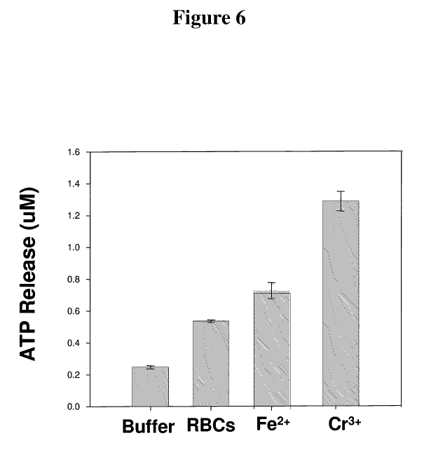

Improving Metal-Induced Activity of C-Peptide. In order to extend the activity of the C-peptide, other metal cations are tested. For example, a chromium (III) source is added to a solution of inactive C-peptide. The data in FIG. 6a show that the Cr(III) is able to bind the C-peptide. The measured mass spectrometric signal of this adduct is found to be more stable than the Fe(II)-C-peptide adduct (cf. FIG. 5). The C-peptide/Cr(III) adduct is also tested for erythrocyte ATP-release bioactivity. FIG. 5b shows that Cr(III) alone does not result in any significant increase in ATP release from deformed RBCs. However, when the C-peptide/Cr(III) adduct is added to a suspension of RBCs, ATP release occurs in a manner similar to that shown in FIG. 4 for the Fe(II)-C-peptide complex.

### Example 6

Extension of C-peptide Activity through Binding to Cr(III). To determine the longevity of bioactive C-peptide/metal ion complexes, aliquots from an inactive solution of the C-peptide are mixed with equimolar amounts of either Fe(II) or Cr(III) and allowed to incubate for 48 h. After this incubation period, the C-peptide/metal cation mixtures are introduced into a fresh RBC suspension and allowed to incubate in the RBCs for 6 h. These cells are then mechanically deformed in order to measure the RBC ATP-release response. The data in FIG. 6a reveals that the activity of the C-peptide bound to Cr(III) results in a significantly greater ATP-release response from the RBCs. Furthermore, the results in FIG. 6b also indicate that the activity of the C-peptide in complex with Cr(III) is extended to periods beyond 4 days. These results for C-peptide/metal cation complex longevity are based on residence times in aqueous solution. However, frozen or lyophilized preparations would generally provide much greater complex longevity, as would preparations, e.g., concentrates, containing an excess of the Cr(III), Fe(II), or other desired polyvalent cation in the presence of little or no monovalent cation content.

### Example 7

Subcutaneous Formulation. To prepare 10 mL of a composition suitable for subcutaneous administration, the following ingredients are combined:

| | |
|---|---|
| Glycerin | 160 mg |
| Human C-peptide/Cr(III)₂ complex | 35 mg |
| Human insulin | 15 mg (28 U/mg) |
| Iridine | 30 mg |
| m-Cresol | 25 mg |

Water and either NaOH or HCl to a final volume of 10 mL with a pH of 6.9-7.8.

To a combination of the C-peptide complex, insulin, and iridine, the insulin is added with vigorous stirring for 30 minutes. To this combination, the m-cresol is added with stirring for an additional 10 minutes; the volume and pH of the mixture is then adjusted. The composition is loaded into a hypodermic syringe and injected subcutaneously. The injection is given 2-4 times per day.

### Example 8

Transdermal Formulation. To prepare a transdermal patch, the following procedure is used:

| | | |
|---|---|---|
| Human C-peptide/Cr(III)₂ complex | 10 | mg |
| Glyburide | 5 | mg |
| Lecithin | 2 | g |
| Refined soybean oil | 6 | mL |

To a mixture of the C-peptide complex and lecithin, the oil is added and the combination is stirred vigorously for 60 minutes. 3 mL of the composition is applied to a 5 cm×5 cm square patch of 3-ply Northern softwood Kraft pulp paper, which had been reinforced with a glyoxylated polyacrylamide wet strength resin. A backing sheet of DURAPORE cloth tape (3M Personal Care Products, St. Paul, Minn.) is provided for use in adhering the transdermal patch to skin. The patch is allowed to remain in place for 4-12 hours.

### Example 9

Enteral Formulation. To prepare capsules for oral administration, the following components are mixed.

| | | |
|---|---|---|
| Aqueous propylene glycol (25% v/v) | 5 | mL |
| Sodium N-[8-(2-hydroxybenzoyl)amino]caprylate | to 50 | mM |
| Lisofylline | 200 | mg |

The combination is stirred for 60 minutes and calcium alginate capsules are loaded with the resulting mixture. 1 capsule is administered 3× per day.

The embodiments and the examples described herein are exemplary and not intended to be limiting in describing the full scope of compositions and methods of the present technology. Equivalent changes, modifications and variations of some embodiments, materials, compositions and methods can be made within the scope of the present technology, with substantially similar results.

What is claimed is:

1. A composition in unit dosage form comprising a pharmaceutically acceptable complex of C-peptide or a fragment thereof with a polyvalent metal cation complex, and a pharmaceutically acceptable carrier.

2. The composition according to claim 1, wherein the C-peptide comprises a mammalian C-peptide amino acid sequence.

3. The composition according to claim 2, wherein the C-peptide comprises the amino acid sequence of a human C-peptide.

4. The composition according to claim 3, wherein the C-peptide comprises the amino acid sequence of SEQ ID NO:2.

5. The composition according to claim 1, wherein the polyvalent metal cation comprises a pharmaceutically acceptable divalent or trivalent metal cation.

6. The composition according to claim 5, wherein the polyvalent metal cation is selected from the group consisting of divalent Mg, Ca, Sr, Ba, Ge, and Sn cations; trivalent Al, Ga, In, and Bi cations; divalent and trivalent transition metal cations; divalent and trivalent La, Ce, Pr, Nd, Pm, Sm, Eu, Gd, Tb, Dy, Ho, Er, Tm, Yb, and Lu cations; and combinations thereof.

7. The composition according to claim 6, wherein the polyvalent metal cation is a transition metal cation.

8. The composition according to claim 7, wherein the polyvalent metal cation is a Cr, Mn, Fe, Co, Ni, Cu, Zn, Mo, Ag, Pt, or Au cation, or a combination thereof.

9. The composition according to claim 8, wherein the polyvalent metal cation is a Cr, Mn, Fe or Zn cation, or a combination thereof.

10. The composition according to claim 9, wherein the polyvalent metal cation is a Cr(III), Fe(II) or Zn(II) cation, or a combination thereof.

11. The composition according to claim 10, wherein the polyvalent metal cation comprises a Cr(III) cation.

12. The composition according to claim 10, wherein the polyvalent metal cation comprises a Zn(II) cation.

13. The composition according to claim 1, wherein the polyvalent metal cation is complexed with at least one aspartate or glutamate side chain of the C-peptide.

14. The composition according to claim 1, wherein the complex comprises from about 10 to about 67 mole percent polyvalent metal cation, based on the total moles of ions present in the complex.

15. The composition according to claim 13, wherein the complexes of the composition collectively comprise from about 25 to about 50 mole percent polyvalent metal cation, based on the total moles of ions present in the complexes.

16. The composition according to claim 1, wherein the complexes comprises a fragment of said C-peptide.

17. A composition according to claim 16, wherein said fragment comprises less than 10 amino acid residues.

18. The composition according to claim 1, further comprising a bioactive material.

19. The composition according to claim 18, wherein the bioactive material comprises a glucose metabolism modulator.

20. The composition according to claim 22, wherein the glucose metabolism modulator comprises insulin.

21. The composition according to claim 1, wherein the composition is suitable for enteral, parenteral, or topical administration.

22. The composition according to claim 21, wherein the composition is a suitable for transdermal, pulmonary, nasal, buccal or ophthalmic administration.

23. The composition according to claim 22, wherein the topical formulation is a powder, a liquid, a gel, or an adhesive article.

24. The composition according to claim 21, wherein the composition is suitable for intravenous, subcutaneous, or intraperitoneal administration.

25. The composition according to claim 24, wherein the composition is a parenteral formulation for intravenous or subcutaneous delivery by hypodermic needle or fluid microjet administration.

26. The composition according to claim 21, wherein the composition is frozen or lyophilized.

27. A method for promoting vasodilation in a human or other animal subject in need thereof, comprising administering to the subject a composition comprising in a pharmaceutically acceptable formulation, a therapeutically effective dose of an erythrocyte ATP-release modulator.

28. The method according to claim 27, wherein the erythrocyte ATP-release modulator is selected from the group consisting of pentoxifylline, lisofylline, epoxidated arachidonic acids, and salts and esters thereof; C-peptides and fragments thereof; mixtures of C-peptide or fragments thereof and a source of a pharmaceutically acceptable polyvalent metal cation; complexes comprising a C-peptide or fragment thereof with a polyvalent metal cation; and mixtures thereof.

29. The method according to claim 28, wherein the erythrocyte ATP-release modulator comprises a complex comprising a C-peptide or fragment thereof with a polyvalent metal cation.

30. The method according to claim 27, wherein said subject has diabetes mellitus type 1, diabetes mellitus type 2, or gestational diabetes.

31. The method according to claim 28, wherein the subject has diabetes mellitus type 1.

32. The method according to claim 31, further comprising administering insulin to the subject.

33. The method according to claim 28, wherein the subject has diabetes mellitus type 2.

34. The method according to claim 33, further comprising administering to the subject an oral hypoglycemic agent.

35. The method according to claim 34, where the oral hypoglycemic agent is selected from the group consisting of tolbutaminde, chlorpropamide, tolazamide, acetohexamide, glyburide, glipizide, gliclazide, and mixtures thereof.

36. The method according to claim 27, wherein the subject has metabolic syndrome, erectile dysfunction, sickle cell anemia, malaria, chronic fatigue syndrome, obesity, a vascular condition, or is undergoing care for a cardiac or stroke condition.

37. The method according to claim 36, wherein the subject has a vascular condition that is hypertension, gestational hypertension, a peripheral vascular disease, chronic venous insufficiency, or Raynaud's disease.

38. The method according to claim 27, wherein said administering comprises administering to the subject a first composition comprising a C-peptide or fragment thereof and administering to the subject a second composition comprising a cation-releasable source of the metal cation.

39. The method according to claim 38, wherein said administering of the first composition and the administering of the second composition are substantially concurrent.

40. The method according to claim 38, wherein the cation-releasable source comprises chromium tripicolinate, chromium citrate, chromium gluconate, chromium aspartate, chromium cysteine, chromium N-acetyl cysteine, chromium trinicotinate, chromium dinicotinate chloride, glucose tolerance factor (GTF, chromium dinicotinoglutathione), chromium trichloride, or a combination thereof.

41. A method for modulating erythrocyte ATP-release response in a human or other animal subject in need thereof, comprising administering the subject a safe and effective amount composition comprising an active selected from the group consisting of pentoxifylline, lisofylline, epoxidated arachidonic acids, and salts and esters thereof; mixtures of C-peptide or a fragment thereof and a source of a pharmaceutically acceptable polyvalent metal cation; complexes comprising a C-peptide or fragment thereof with and a polyvalent metal cation; and mixtures thereof.

42. A method for promoting serum glucose clearance in a human or animal subject in need thereof, comprising administering to the subject a composition comprising a safe and effective amount of an erythrocyte ATP-release modulator.

43. The method according to claim 42, wherein said subject has a glucose processing disorder.

44. The method according to claim 43, wherein the glucose processing disorder is diabetes mellitus type 1, diabetes mellitus type 2, or gestational diabetes.

45. The method according to claim 43, wherein the glucose processing disorder is metabolic syndrome.

46. The method according to claim 42, wherein the erythrocyte ATP-release modulator is selected from the group consisting of pentoxifylline, lisofylline, epoxidated arachidonic acids, and salts and esters thereof; C-peptides and fragments thereof; mixtures of C-peptide or fragments thereof and a source of a pharmaceutically acceptable polyvalent metal cation; complexes comprising a C-peptide or a fragment thereof with a polyvalent metal cation; and mixtures thereof.

47. A method for promoting glucose clearance or vasodilation in a human or animal subject, comprising administering to the subject a safe and effective amount of a pharmaceutically acceptable C-peptide/metal cation complex in which the metal cation comprises a pharmaceutically acceptable M(II) or M(III) cation.

48. The method according to claim 47, wherein the composition is administered as a prophylactic treatment to a subject identified as being at risk for developing a disorder of glucose processing.

49. The method according to claim 48, wherein the composition is administered as a palliative treatment for a glucose processing disorder.

50. The method according to claim 47, further comprising administering to said subject a glucose metabolism modulator.

51. The method according to claim 50, wherein said glucose metabolism modulator is insulin.

52. The method according to claim 50, wherein said glucose metabolism modulator is an oral hypoglycemic agent.

53. The method according to claim 52, wherein said glucose metabolism modulator is selected from the group consisting of tolbutaminde, chlorpropamide, tolazamide, acetohexamide, glyburide, glipizide, gliclazide, and mixtures thereof.

54. A regimen for treating diabetes mellitus in a human or other animal subject comprising administering to the subject a safe and effective amount of a glucose metabolism modulator and a safe and effective amount of an erythrocyte ATP-release modulator, wherein said erythrocyte ATP-release modulator is effective to reduce the level of the glucose metabolism modulator needed to effect glucose control in the subject, extend the duration of efficacy of the glucose metabolism modulator in the subject, or both.

55. The regimen according to claim 54 for treatment of diabetes mellitus Type 1, wherein the glucose metabolism modulator is insulin.

56. The regimen according to claim 54 for treatment of diabetes mellitus Type 2, wherein the glucose metabolism modulator is an oral hypoglycemic agent.

57. A process for preparing a C-peptide/polyvalent metal cation complex, comprising:
(A) providing an isolated or recombinant C-peptide or fragment thereof and a source of a pharmaceutically acceptable polyvalent metal cation; and
(B) contacting the peptide and cation source under conditions in which the cation can attach to acidic residues of the peptide, thereby forming a complex.

58. A kit for the preparation of a C-peptide/polyvalent metal cation complex, the kit comprising a frozen or lyophilized C-peptide or fragment thereof, and a source of a pharmaceutically acceptable polyvalent metal cation, with instructions for preparing a C-peptide/polyvalent metal cation complex therefrom, optionally further comprising instructions for making a pharmaceutical formulation containing the complex, and optionally further comprising instructions for administering the formulation to a human or animal subject.

59. A composition for the treatment of glucose metabolism disorders, comprising a C-peptide/polyvalent metal cation complex; a glucose metabolism modulator; and a pharmaceutically acceptable carrier.

60. A composition for the treatment of glucose metabolism disorders, comprising a glucose metabolism modulator and a cation-releasable material.

61. The composition of claim 60, wherein the cation-releasable material comprises chromium tripicolinate, chromium citrate, chromium gluconate, chromium aspartate, chromium cysteine, chromium N-acetyl cysteine, chromium trinicotinate, chromium dinicotinate chloride, glucose tolerance factor (GTF, chromium dinicotinoglutathione), chromium trichloride, or a combination thereof.

62. The composition of claim 60, wherein the glucose metabolism modulator is selected from the group consisting of insulin, hypoglycemic agents, and mixtures thereof.
